(19) United States
(12) Patent Application Publication
(10) Pub. No.: US 2026/0182890 A1
(43) Pub. Date: Jul. 2, 2026

(54) SLEEVE MOUNTING DEVICE FOR BASKET DISTAL END ASSEMBLY

(71) Applicant: BIOSENSE WEBSTER (ISRAEL) LTD., Yokneam (IL)

(72) Inventors: Assaf Govari, Haifa (IL); Elad Avraham Diukman, Haifa (IL); Stanislav Katzir, Hadera (IL); Nikolay Veits, Afula (IL); Sergey Sobol, Haifa (IL)

(21) Appl. No.: 19/007,966

(22) Filed: Jan. 2, 2025

(51) Int. Cl. A61B 5/287; A61B 5/00

(52) U.S. Cl. CPC A61B 5/287; A61B 5/6858; A61B 2562/12

(57) ABSTRACT

An apparatus for manufacturing an end effector for a basket catheter assembly includes a support platform and a force application mechanism. The support platform is configured to support an expandable basket formed of curved splines with shape retention. Guidelines pass through longitudinal channels of respective flexible sleeves and are connected to the free end of respective splines. The force application mechanism controllably applies and releases a tensile force on the second end of the guidelines. Pulling on a guideline applies a tensile force to the free end of the spline, thereby flattening it. This enables sliding the flattened spline through the longitudinal channel of the flexible sleeve. The spline reverts to a curved shape when the tensile force is released.

### TECHNOLOGICAL FIELD

The presently disclosed subject matter generally relates to the field of therapeutic basket catheters, and, more particularly, but not exclusively, to assembly of the distal end of a basket catheter.

### BACKGROUND

Cardiac arrhythmias, such as atrial fibrillation (AF), occur when regions of cardiac tissue abnormally conduct electric signals to adjacent tissue. This disrupts the normal cardiac cycle and causes asynchronous rhythm. Certain procedures exist for treating arrhythmia, including surgically disrupting the origin of the signals causing the arrhythmia and disrupting the conducting pathway for such signals. By selectively ablating cardiac tissue by application of energy via a catheter, it is sometimes possible to cease or modify the propagation of unwanted electrical signals from one portion of the heart to another. Some ablation approaches use irreversible electroporation (IRE) to ablate cardiac tissue using nonthermal ablation methods. IRE delivers short pulses of high voltage to tissues and generates an unrecoverable permeabilization of cell membranes

Regions of cardiac tissue can be mapped by a catheter to identify the abnormal electrical signals. The same or different catheter can be used to perform ablation. Some example catheters include a distal end effector formed from a number of splines with electrodes positioned thereon.

### OVERVIEW

The present disclosure addresses the technical challenges associated with manufacturing catheters for use in catheter-based electrophysiology, particularly for mapping and ablation procedures. Electrophysiology catheters typically include an expandable end effector including flexible splines forming, in its expanded form, a basket shape. Flexible sleeves (typically tubular) carrying electrodes encircle the splines.

It is desirable that the flexible sleeve fit relatively tightly around the spline, so that it remains in the desired location during the bonding process. This makes it difficult to slide the spline through the sleeve.

To address this issue, aspects according to the present disclosure provide an apparatus and method for manufacturing an end effector for a basket catheter assembly. The method involves applying a tensile force to the splines to flatten them, allowing for the easy insertion of the splines into flexible sleeves that include electrodes. Once the flexible sleeves are positioned, the tensile force is released, allowing the splines to revert to their curved shape with the sleeve in place. In this manner the manufacturing process of the end effector is simplified, reducing time and cost for manufacturing both the end effector and the entire basket catheter assembly.

Reference is now made to FIG. 1A, which is a schematic view of a catheter assembly 100, in accordance with examples of the present disclosure.

The catheter assembly 100 includes an expandable basket 120 comprising shape-set flexible splines 130 connected together (only some labeled for the sake of simplicity) at the distal end 140 of expandable basket 120. Expandable basket 120 is coupled to the distal end of shaft 110. Shaft 110 is typically inserted into a delivery sheath (not shown in FIG. 1) so as to be guided to the desired location. An operator (e.g. a physician) may place expandable basket 120 of the catheter 100 in contact with the epicardial tissue surface for sensing and/or ablating.

As used herein, with respect to a spline, the term “shape-set” means that when a force applied to a shape-set spline it may change its curvature, and the spline returns to its set shape when the force is released.

Flexible plastic sleeves 150 encircle some or all of the length of respective splines 130. Each plastic sleeve 150 has at least one electrode 151-153 positioned along its outer surface. Electrodes 151-153 may be configured for delivering ablation energy such as Radio Frequency (RF) and/or Pulse Field Ablation (PFA) to cardiac tissue and/or for sensing electrical signals (e.g. the location of expandable basket 120 and/or to measure a physiological property such as local surface electrical potentials at respective locations on cardiac tissue).

In some examples, expandable basket 120 is configured to transition from the collapsed form to the expanded form by release of radial constraints on the flexible splines 130, for example when pushed out of a delivery sheath. The flexible splines 130 may also retract when subjected to radial constraints, for example when pulled into the delivery sheath.

Catheter assembly 100 may further include coupler 160, which couples the proximal ends of splines 130 to the distal end of tubular shaft 110.

Splines 130 may be formed of any suitable material, for example, but not limited to, shape-set Nitinol. In some examples, expandable basket 120 is formed as single unit, for example from a single piece of shape-set Nitinol.

Reference is now made to FIG. 1B, which is a longitudinal cross section view of a sleeve assembly, in accordance with an example of the present disclosure. Sleeve assembly 170 includes a flexible plastic sleeve 150 having a longitudinal channel 180 forming a conduit through which a spline may be inserted. Three ring-shaped electrodes 151-153 encircle plastic sleeve 150. Wires 190 are connected to a flexible printed circuit board (fPCB) 191 embedded in plastic sleeve 150 and supply charge to respective electrodes 151-153. Wires 190 may connect to fPCB 191, inside or outside plastic sleeve 150. Optionally, one face of fPCB 191 is exposed within longitudinal channel 180.

Reference is now made to FIG. 1C, which is a transverse cross section view of a sleeve assembly, in accordance with an example of the present disclosure. Sleeve assembly 170 includes plastic sleeve 150 having a longitudinal channel 180. Ring-shaped electrodes 151-153 encircle plastic sleeve 150. Wires 190 connect to fPCB 191 within plastic sleeve 150 and connect to electrodes 151-153 respectively.

### BRIEF DESCRIPTION OF THE DRAWINGS

In order to better understand the subject matter that is disclosed herein and to exemplify how it may be carried out in practice, examples will now be described, by way of non-limiting example only, with reference to the accompanying drawings, in which:

FIG. 1A is a schematic view of a catheter assembly, in accordance with an example of the present disclosure;

FIG. 1B is a longitudinal cross section view of a sleeve assembly, in accordance with an example of the present disclosure;

FIG. 1C is a transverse cross section view of a sleeve assembly, in accordance with an example of the present disclosure;

FIGS. 2-4 are schematic drawings of an apparatus for manufacturing an end effector for a basket catheter assembly, according to a first example of the disclosure;

FIG. 5 is a schematic drawing of an apparatus for manufacturing an end effector for a basket catheter assembly, according to a second example of the disclosure;

FIGS. 6A-6B are simplified top views of sub-assemblies for manufacture into an end effector of a basket catheter assembly, according to respective aspects of the disclosure;

FIG. 7 is a simplified flowchart of a method of manufacturing an end effector for a basket catheter assembly, according to some aspects of the disclosure; and

FIG. 8 is a simplified flowchart of a method for applying a tensile force to the free end of a spline, according to an example of the disclosure.

### DETAILED DESCRIPTION

In the following detailed description, numerous specific details are set forth in order to provide a thorough understanding. However, it will be understood by those skilled in the art that the presently disclosed subject matter may be practiced without these specific details. In other instances, well-known methods and features have not been described in detail so as not to obscure the presently disclosed subject matter.

### I. Apparatus for Manufacturing an End Effector

According to some aspects of the disclosure, an apparatus for manufacturing an end effector for a basket catheter assembly includes a support platform and a force application mechanism.

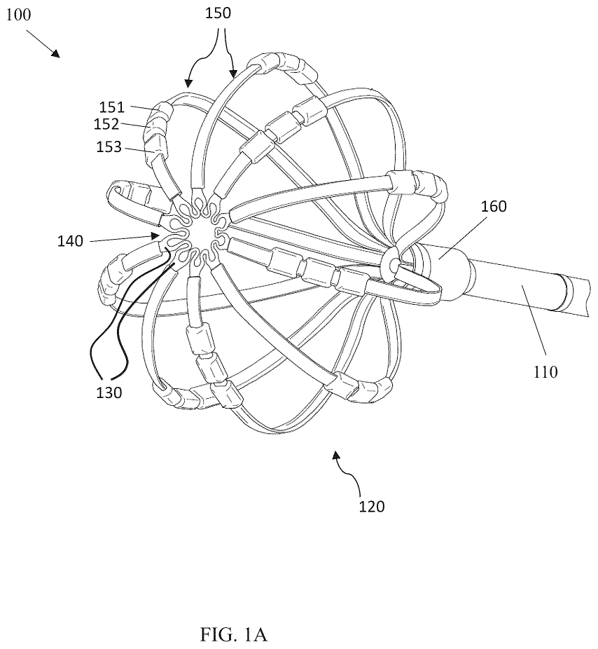

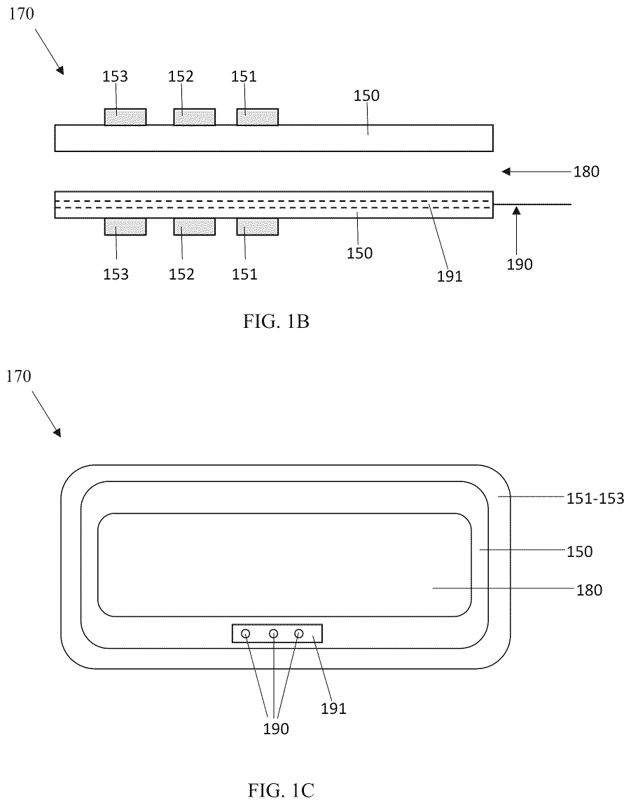

The support platform supports an expandable basket of a basket catheter assembly and multiple sleeve assemblies. The expandable basket includes multiple curved splines with shape retention. The expandable basket is typically configured substantially as shown and described with respect to FIGS. 1A-1C, though specific aspects such as the number of splines, spline curvature, interconnection shape at the top of the expandable basket and so forth may differ for different types of expandable baskets.

Each sleeve assembly includes a flexible sleeve and at least one ring-shaped electrode encircling flexible sleeve. A hollow channel runs longitudinally through the flexible sleeve. During manufacture each spline will be flattened, slid through the longitudinal channel and then released to return to its set shape.

Guidelines are threaded through the hollow channels of respective flexible sleeves. One end of each guideline is releasably connected to the free end of a respective spline. The second ends are connected to a force application mechanism. The force application mechanism controllably applies and releases a tensile force on the guidelines which convey the tensile force to respective free ends of one or more splines. When the tensile force is applied the spline is flattened, thereby enabling or easing sliding the flattened spline through the hollow channel of a flexible sleeve. Each flattened spline reverts to its set shape when the respective tensile force is released. An end effector suitable for ablation procedures is obtained after flexible sleeves have been placed on all the desired splines.

As used herein, the term “free end of a spline” means the proximal end of the spline (before the splines are coupled by a coupler such as 160 in FIG. 1).

In one aspect of the disclosure, support platform 210 includes seats which support respective sleeve assemblies and maintain them in the correct location as the spline is flattened.

In a first example, the force application mechanism includes multiple components, each of which applies a tensile force to a respective spline. In a second example, the force application mechanism is a single component that applies a tensile force to multiple splines (such as a single motor that simultaneously pulls on multiple guidelines connected to respective splines).

According to some aspects of the disclosure, the force application mechanism applies and releases a tensile force on multiple splines simultaneously. Respective flexible sleeves may be slid over the flattened splines before the tensile force is released.

According to some alternate aspects of the disclosure, the force application mechanism applies and releases a tensile force to a single spline at a time, and the splines are flattened in turn.

According to some aspects of the disclosure, the apparatus for manufacturing an end effector further includes a compression mechanism which controllably applies a vertical force to press the top of the expandable basket (e.g. distal end 140 in FIG. 1) towards the support platform. Applying a downward force from the top of the basket may stabilize the location of the expandable basket on the support platform and/or reduce the amount of tensile force that is required to flatten the splines. The compression mechanism may be operated manually or using a motor or other element (e.g. a z-stage). Pressing the top of the basket towards the support platform may be advantageous for flattening all the splines at the same time.

According to some alternate or additional aspects of the disclosure, the top of the expandable basket is supported by a basket support element, such as a clamp or similar device. Stabilizing the top of the basket using the basket support element may be particularly advantageous for flattening single splines (as opposed to all splines together), as the spline is not necessarily flattened onto a support platform. Instead the flattened spline may extend outward from the stabilized top of the basket.

According to some aspects of the disclosure, the support platform is configured to rotate around a central axis. This provides the advantage that a person or device sliding the flexible sleeve onto the spline, as there is no need to move to a new position for each spline. Instead the spline itself may be rotated to a convenient direction.

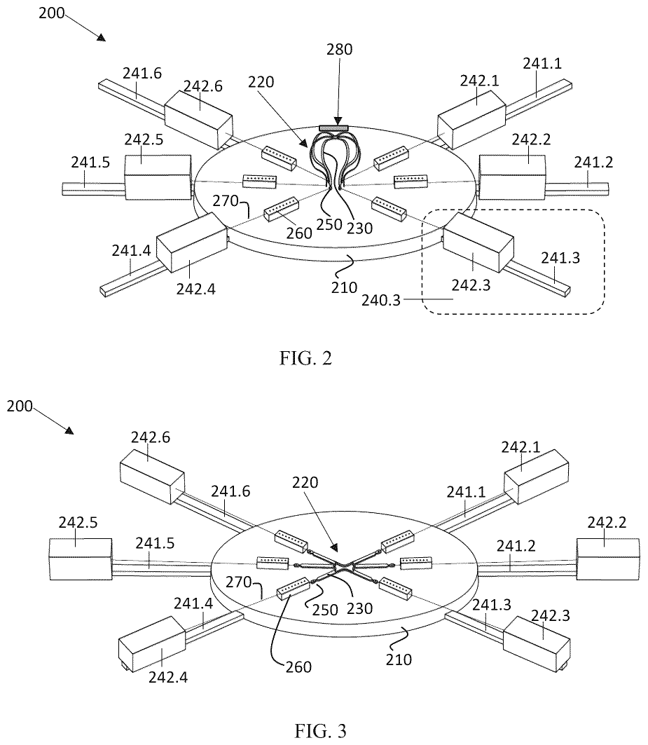

In some aspects of the disclosure, at least a part of the force application mechanism is fixedly coupled to the support platform (e.g. support rails 240.1-241.6 in the example of FIG. 2). In a further aspect the fixedly coupled part of the force application mechanism is configured to rotate together with the support platform.

In some aspects of the disclosure, the apparatus further includes a heat source which controllably heats the flexible sleeve. Including a heat source in the apparatus is particularly useful when the flexible sleeve is configured to contract upon the application of heat (e.g. the flexible sleeve includes heat-shrink tubing). The applied heat causes the flexible sleeve to shrink around the spline, thereby securing the sleeve in place.

In an additional or alternate example, the flexible sleeve is secured on the spline by an adhesive.

The apparatus for manufacturing an end effector for a basket catheter assembly thus enables manipulating the splines of an expandable basket for a basket catheter in a way that facilitates their insertion into flexible sleeves, thus providing a simpler and more time efficient way of accurately manufacturing the end effector.

### I.1. Apparatus for Manufacturing an End Effector Having a Motorized Force Application Mechanism

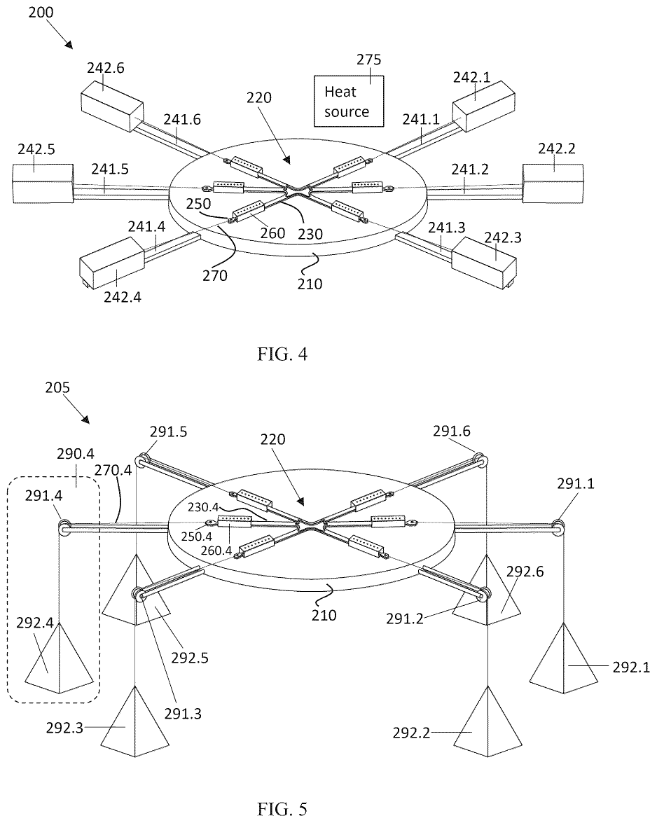

Reference is now made to FIGS. 2-4 which are schematic drawings of an apparatus for manufacturing an end effector for a basket catheter assembly, according to a first example of the disclosure. FIGS. 2-4 illustrate the apparatus at different stages of the manufacturing process. FIG. 2 shows an initial stage before a tensile force has been applied to the splines. FIG. 3 shows the splines after tensile forces have flattened them on a support platform but the flexible sleeves are still on the guidelines. FIG. 4 shows the flattened splines within respective flexible sleeves.

Apparatus 200 includes support platform 210, which is configured to support an expandable basket 220. Expandable basket 220 includes curved splines 230 (only one marked for clarity). The splines possess shape retention characteristics, allowing them to maintain their curved shape unless acted upon by an external force.

FIGS. 2-4 show a non-limiting example of an expandable basket 220 comprising six splines 230. As will be apparent to the skilled person, the number of splines, and consequently the corresponding parts of apparatus 200, may be greater or smaller than six.

Apparatus 200 includes a force application mechanism having multiple components. In the non-limiting example of FIGS. 2-4, each component k of the force application mechanism includes a respective support rail 241.k and motor 242.k which is configured to travel back and forth along the support rail (for example component 240.3 includes support rail 241.3 and motor 242.3 as described below). The tensile force is applied when motor travels outwards on the support rail and is released when the motor travels inwards toward support platform 210.

In an alternate example, the components travel along slits within the support platform.

Each force application mechanism component is configured to controllably apply and release a tensile force to a free end 250 (only one marked for clarity) of a respective spline. When the tensile force is applied to free end 250, spline 230 is flattened. This flattened state facilitates the insertion of the spline 230 through a longitudinal channel within a respective flexible sleeve 260 (only one marked for clarity). The flexible sleeve 260 has at least one electrode positioned along the length of its outer surface.

For clarity, in FIGS. 2-6B the sleeve assembly electrodes are illustrated in a non-limiting manner as dots on the top surface of flexible sleeve 260. In other aspects according to the disclosure, the electrode(s) may have a different shape and/or size relative to the flexible sleeve, such as the ring-like shape of 151-153 in FIGS. 1A-1C.

Upon releasing the tensile force, the flattened spline 230 reverts to its original curved shape, taking advantage of its shape retention properties. This capability ensures that the spline 230 can return to its functional form once it is positioned within the flexible sleeve 260, thus enabling the assembly of the end effector in a controlled and precise manner.

While spline 230 is in a flattened state, flexible sleeve may be slid over a guideline 270 and onto spline 230. Once the flexible sleeve is in the correct location, guideline 270 is released from the free end and the spline returns to its set shape. Guideline 270 may be a wire extending through longitudinal channel 180 and connected to free end 250 of expandable basket 220 on one end or a core insert that is positioned through passage 180. The core insert when used is latched on to free end 250 of expandable basket 220 on one end and to a movable member at an opposite end. The movable member pulls core insert so that as the core insert is extracted out of passage 180, free end 250 of expandable basket 220 is pulled into longitudinal channel 180.

In one example, the location that the flexible sleeve should be positioned at on the spline is indicated by a marking (for example on the support platform or the spline itself).

In an alternate or additional example, the flexible sleeve is physically prevented from being positioned too far up the sleeve (for example by a protrusion on the spline).

In a further example, prior to releasing guideline 270, the force application mechanism controllably loosens guideline 270 and permits it to reduce the tensile force to spline 230 or eliminate the tensile force altogether. Guideline 270 may thus be released from free end 250 after spline 230 has returned to its set shape.

According to some aspects of the disclosure, apparatus 200 further includes a compression mechanism and/or support element 280 (shown only in FIG. 2 for clarity). Compression mechanism and/or support element 280 is in physical contact with the top of expandable basket 230.

In some examples, element 280 acts as a compression mechanism which controllably applies a vertical force to press the top of the expandable basket (e.g. distal end 140 in FIG. 1) towards support platform 200. According to some additional or alternate examples, element 280 acts as a support element that supports the top of the expandable basket, for example by clamping it between two surfaces.

According to an aspect of the disclosure, the splines comprise shape-set nitinol.

### I.2. Apparatus for Manufacturing an End Effector Having a Weight and Pulley Force Application Mechanism

According to some aspects of the disclosure, the force application mechanism includes one or more pulleys and a weight control mechanism. Each pulley is configured to guide a guideline which has one end releasably connected to the free end of a respective spline and a second end connected to a respective weight. The guideline passes through a longitudinal channel of flexible sleeve having one or more electrodes on an outer surface, as described above.

The weight control mechanism controls the motion of the weight(s). Lowering a weight in a controlled manner and/or allowing it to drop downwards applies a tensile force to the end of the spline, thereby flattening the spline. Raising the weights reduces the tensile force on the end of the spline.

In a first example, the weight control mechanism includes one or more actuators, where each actuator holds a guideline in a fixed position until it is time to apply the tensile force to the spline. When it is time to apply the tensile force to the spline, the actuator releases the guideline.

In a second example, the weight control mechanism includes a shelf or other surface on which the weight(s) are placed. A motor (or similar device) moves the shelf up and down, thereby applying and releasing a tensile force simultaneously to the splines connected to the weights.

Reference is now made to FIG. 5, which is a schematic drawing of an apparatus for manufacturing an end effector for a basket catheter assembly, according to a second example of the disclosure. In the instant example, the apparatus has a force application mechanism which includes at least one weight and at least one pulley. FIG. 5 illustrates a stage of the manufacturing process in which the splines have been flattened by the tensile force and the flexible sleeves have been positioned on the respective splines.

Apparatus 205 includes support platform 210, which is configured to support an expandable basket 220. Expandable basket 220 includes curved splines 230 (only one marked for clarity). These splines possess shape retention characteristics, allowing them to maintain their curved shape unless acted upon by an external force.

In the example of FIG. 5, the force application mechanism includes multiple components, each of which applies a tensile force to a respective spline via the respective guideline. Each component of the force application mechanism includes a respective pulley and weight. The guideline is positioned over the pulley, so that downward movement of the weight is transformed into a tensile force on the free end of the spline.

For example component 290.4 includes pulley 291.4 and weight 292.4. When weight 292.4 moves downward a tensile force is applied by guideline 270.4 to the free end of spline 230.4.

### II. Sub-Assembly for Manufacturing an End Effector

According to some aspects of the disclosure, a sub-assembly for manufacturing into an end effector of a basket catheter assembly includes an expandable basket, sleeve assemblies and guidelines. The expandable basket is formed from multiple curved splines with shape retention. The splines are connected together at a top of the expandable basket. Each sleeve assembly includes a flexible sleeve with one or more ring-shaped electrodes encircling it. The guidelines are releasably connected to the free ends of respective splines, for example by tying the guideline to a hole in the free end of the spline. Each guideline is threaded through a respective flexible sleeve, substantially as described above.

During the manufacturing process, the guidelines may serve to convey a tensile force to the free end of the respective spline.

In one aspect of the disclosure, the second end of each of the guidelines is connected to a force application mechanism which is configured to apply and release a force on the guidelines. The guidelines may be connected to the force application mechanism in any suitable way (e.g. tied, clipped, glued, etc...). In an example, each guideline is connected to a respective component of the force application mechanism.

In one example, the second end of each of the guidelines is attached to a respective weight.

According to an aspect of the disclosure, the splines are shape-set nitinol.

According to an aspect of the disclosure, the flexible sleeves are made of a material that contracts upon the application of heat. Thus, heating the flexible sleeve causes it to shrink, securing its location on the spline.

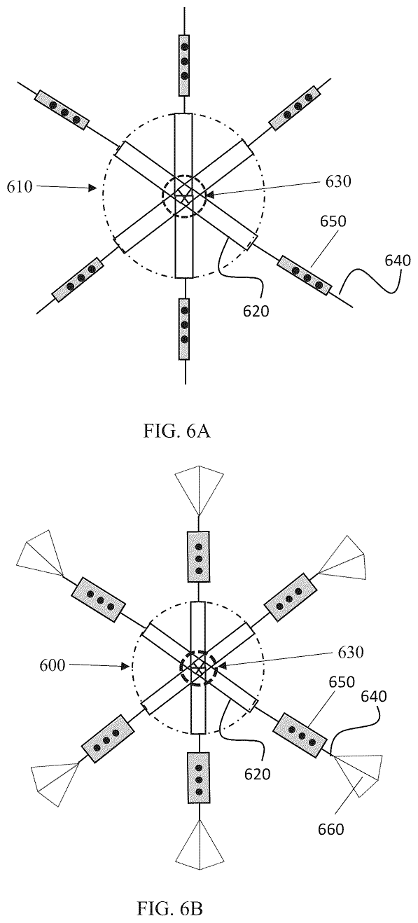

Reference is now made to FIGS. 6A-6B, which are simplified top views of sub-assemblies for manufacture into an end effector of a basket catheter assembly, according to respective aspects of the disclosure. The sub-assembly is configured to be placed as a unit upon a support platform.

In the instant examples, the sub-assembly includes an expandable basket (indicated by a dashed circle 610). Expandable basket 610 includes multiple splines 620 (only one marked for clarity). The splines are joined together at the top of the expandable basket (indicated by dashed circle 630). The free end of each spline is connected to a respective guideline 640 which runs through a respective flexible sleeve 650.

FIG. 6A illustrates a first example in which the guidelines are connected at one end to respective splines and the second end is free. The free end may later be connected to a force application mechanism or components thereof (not shown).

FIG. 6B illustrates a second example in which the guidelines are connected at one end to respective splines and the second end is connected to a respective weight 660 (only one marked for clarity). During manufacture the sub-assembly may be placed on the support platform and the guidelines are run over the pulleys.

### III. Method for Manufacturing an End Effector

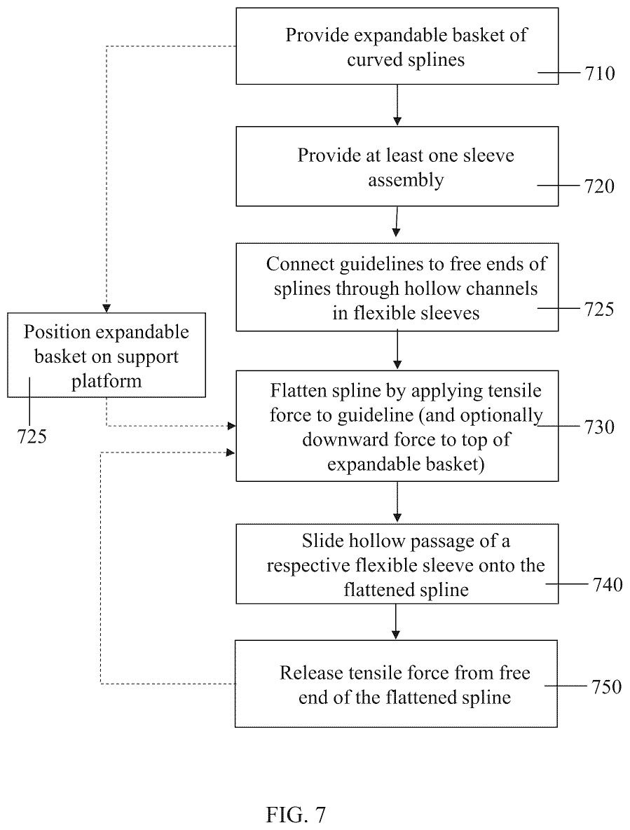

Reference is now made to FIG. 7, which is a simplified flowchart of a method of manufacturing an end effector for a basket catheter assembly, according to some aspects of the disclosure.

In 710, an expandable basket which includes multiple curved splines is provided. The splines have shape retention properties as described above.

In 720, a respective sleeve assembly is provided for at least one spline. Each sleeve assembly includes a flexible sleeve with a longitudinal channel and at least one ring-shaped electrode encircling the flexible sleeve.

In 725, guidelines running through channels in the flexible sleeves are releasably connected the free ends of respective splines.

In 730-750, the flexible sleeve(s) are positioned on the spline(s). In 730 a tensile force is applied to the free end of the spline by applying a force to the respective guideline, thereby flattening its curved shape. In 740, once flattened, the longitudinal channel of the respective flexible sleeve is slid onto the spline. In 750 the tensile force is released, allowing the spline to revert to its original curved shape.

In one example, when flexible sleeves are to be positioned on multiple splines, 730-750 may be repeated for each spline (as indicated by the dashed arrow from 750 to 730). In an alternate example, multiple splines may be flattened and released at the same time, so that respective flexible sleeves may be positioned on more than one spline between the flattening and releasing processes.

In a further aspect, in 725 the expandable basket is positioned on a support platform.

According to some aspects, a respective guideline is releasably connected to the free end of at least one spline. The guideline passes through the longitudinal channel of the respective flexible sleeve having electrode(s) on its outer surface. The tensile force is applied to the free end of the spline by pulling on the guideline. When enough force is applied, the spline is flattened, facilitating sleeve application. In a further aspect, the releasable connection is achieved by tying the end of the guideline to a hole in the free end of the spline.

In some aspects of the disclosure, tensile forces are applied simultaneously to multiple splines, allowing for concurrent sleeve placement on multiple splines of the expandable basket.

In an example, the magnitude of the applied tensile force is sufficient to flatten splines comprised of shape-set nitinol.

In some aspects of the disclosure, a downward force is controllably applied to the top of the expandable basket to control its position on the support platform. The downward force may also assist in flattening the spline(s), since a lower magnitude tensile force may be required when force is being applied from above.

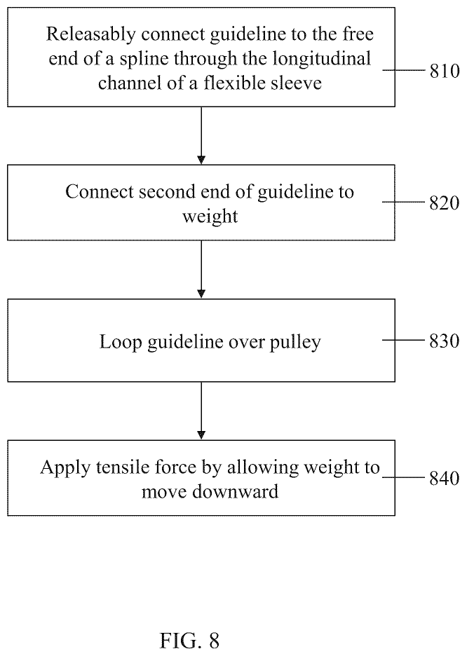

Reference is now made to FIG. 8, which is a simplified flowchart of a method for applying a tensile force to the free end of a spline, according to an example of the disclosure. Tensile forces are applied to the splines by the weight and pulley technique described above.

In 810, the first end of a guideline is releasably connected to the free end of a spline through the longitudinal channel of a flexible sleeve having electrode(s) on an outer surface.

In 820, the second end of the guideline is connected to a weight.

In 830, the guideline is looped over a pulley, while securing the weight against downward motion.

In 840, the weight is allowed to move downwards so as to apply the tensile force to the free end of the at least one spline.

In order to position flexible sleeves on more than one spline, 810-840 may be performed sequentially for each spline. Alternately, multiple splines may be flattened by allowing multiple weights to move downward simultaneously, where each weight is connected by a guideline to a respective spline.

Following is a non-exclusive list of some exemplary examples of the disclosure. The present disclosure also includes examples which include fewer than all the features in an example and examples using features from multiple examples, even if not listed below.

### Example 1

An apparatus 200 for manufacturing an end effector for a basket catheter assembly, comprising:
a support platform 210, configured to support an expandable basket 220 comprising a plurality of curved splines 230 with shape retention and a plurality of sleeve assemblies, each of the sleeve assemblies respectively comprising a flexible sleeve 260 and at least one ring-shaped electrode 151-153 encircling the flexible sleeve 260;
a plurality of guidelines 270, each of the guidelines 270 passing through a longitudinal channel 180 of a respective flexible sleeve 260 and having a first end releasably connected to a free end of a respective spline 230 and a second end;
a force application mechanism associated with the support platform 210, configured to controllably apply and release a tensile force to the second ends of the guidelines 270 such that a spline 230 is flattened when the tensile force is applied to the second end of the respective guideline 270 and reverts to a curved shape when the tensile force is released, so as to enable sliding the flattened spline 230 through the longitudinal channel 180 of the respective flexible sleeve 260.

### Example 2

The apparatus 200 of Example 1, wherein the force application mechanism comprises:
at least one pulley 291.4, configured to guide a respective guideline 270.4 having second end connected to a respective weight 292.4; and
a weight control mechanism, configured to control at least downward motion of the weight 292.4, such that the guideline 270.4 applies the tensile force to the free end 250.4 of the respective spline 230.4 as the weight descends.

### Example 3

The apparatus 200 of Example 1 or Example 2, wherein the support platform 210 is configured to rotate around a central axis.

### Example 4

The apparatus 200 of any one of Examples 1-3, wherein the force application mechanism is fixedly coupled to the support platform 210.

### Example 5

The apparatus 200 of any one of Examples 1-4, further comprising a compression mechanism 280, configured for controllably applying a vertical force to press a top of the expandable basket 220 towards the support platform 210.

### Example 6

The apparatus 200 of any one of Examples 1-5, further comprising a basket support element 280 for supporting a top of the expandable basket 220 while the tensile force is applied.

### Example 7

The apparatus 200 of any one of Examples 1-6, wherein the flexible sleeve 260 is configured to contract upon the application of heat, the apparatus 200 further comprising a heat source 275 configured for applying heat to the flexible sleeve 260 so as to secure the flexible sleeve 260 to a spline 230 passing through the flexible sleeve 260.

### Example 8

The apparatus 200 of any one of Examples 1-7, wherein the support platform 210 comprises seats for supporting respective sleeve assemblies.

### Example 9

A sub-assembly for manufacture into an end effector of a basket catheter assembly, comprising:
an expandable basket 220 comprising a plurality of curved splines 620 with shape retention;
a plurality of sleeve assemblies, each of the sleeve assemblies respectively comprising a flexible sleeve 650 having a longitudinal channel 180 therethrough and at least one ring-shaped electrode 151-153 encircling the flexible sleeve 650; and
a plurality of guidelines 640 threaded through the longitudinal channels 180 of respective sleeve assemblies, each of the guidelines 640 being releasably connected to a free end of a respective spline 620 and configured to apply a tensile force to the free end of the respective spline 620.

### Example 10

The sub-assembly of Example 9, wherein a second end of each of the guidelines 640 is attached to a respective weight 660.

### Example 11

The sub-assembly of Example 9 or Example 10, wherein a second end of each of the guidelines 640 is connected to a force application mechanism configured to apply and release a tensile force on the guidelines 640.

### Example 12

The sub-assembly of any one of Examples 9-11, wherein the splines 620 comprise shape-set nitinol.

### Example 13

The sub-assembly of any one of Examples 9-12, wherein the flexible sleeves 650 are configured to contract upon the application of heat.

### Example 14

A method of manufacturing an end effector for a basket catheter assembly, comprising:
providing an expandable basket 220 comprising a plurality of curved splines 230 with shape retention;
providing at least one sleeve assembly, each of the sleeve assemblies respectively comprising a flexible sleeve 260 and at least one ring-shaped electrode 151-153 encircling the flexible sleeve 260;
releasably connecting a first end of a guideline 270 to a free end of a respective spline 230 through a longitudinal channel 180 of a flexible sleeve 260, such that pulling on the guideline 270 applies a tensile force to the free end of the respective spline 230; and
for at least one of the splines 230:
flattening the spline 230 by applying a tensile force to a second end of a respective guideline 270;
sliding a longitudinal channel 180 of a respective flexible sleeve 260 onto the flattened spline 230; and
releasing the tensile force from the second end of the guideline 270 to allow the spline 230 to revert to a curved shape.

### Example 15

The method of Example 14, wherein the releasably connecting the guideline 270 to the free end 250 of the at least one spline 230 comprises tying an end of the guideline 270 to a hole in the free end 250 of the spline 230.

### Example 16

The method of any one of Examples 14-15, further comprising:
connecting a second end of the guideline 270.4 to a weight 292.4;
looping the guideline 270.4 over a pulley 291.4 while securing the weight 292.4 against downward motion; and
moving the weight 292.4 downwards so as to apply the tensile force to the free end 250.4 of the at least one spline 230.4.

### Example 17

The method of any one of Examples 14-16, wherein a tensile force is applied simultaneously to at least some of the plurality of splines 230.

### Example 18

The method of any one of Examples 14-17, further comprising positioning the basket 220 on a support platform 210.

### Example 19

The method of Example 18, further comprising controllably applying a downward force on a top of the expandable basket 220 so as to control a position of the top of the expandable basket 220 relative to the support platform 210.

### Example 20

The method of any one of Examples 13-19, further comprising supporting a top of the expandable basket 220 in place while the tensile force is applied.

### Example 21

The method of any one of Examples 13-20, wherein a magnitude of the tensile force is sufficient to flatten a spline 230 comprising shape-set nitinol.

Those skilled in the art to which the present disclosure pertains, can appreciate that while the present disclosure has been described in terms of preferred examples, the concept upon which this disclosure is based may readily be utilized as a basis for the designing of other structures, systems and processes for carrying out the several purposes of the present disclosure.

Also, it is to be understood that the phraseology and terminology employed herein are for the purpose of description and should not be regarded as limiting. It should be noted that the words “comprising”, “including” and “having” as used throughout the appended claims are to be interpreted to mean “including but not limited to”. The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases, and disjunctively present in other cases. The term “each” may not be exclusively understood as referring to each and every, and when technically relevant may also refer to “at least some”.

All patents and patent applications mentioned in this specification are herein incorporated in their entirety by reference into the specification, to the same extent as if each individual patent or patent application was specifically and individually indicated to be incorporated herein by reference. In addition, citation or identification of any reference in this application shall not be construed as an admission that such reference is available as prior art to the present disclosure.

It is important, therefore, that the scope of the present disclosure is not construed as being limited by the illustrative examples set forth herein. Other variations are possible within the scope of the present disclosure as defined in the appended claims. Other combinations and sub-combinations of features, functions, elements and/or properties may be claimed through amendment of the present claims or presentation of new claims in this or a related application. Such amended or new claims, whether they are directed to different combinations or directed to the same combinations, whether different, broader, narrower or equal in scope to the original claims, are also regarded as included within the subject matter of the present description.

1. An apparatus for manufacturing an end effector for a basket catheter assembly, comprising:
a support platform, configured to support an expandable basket comprising a plurality of curved splines with shape retention and a plurality of sleeve assemblies, each of said sleeve assemblies respectively comprising a flexible sleeve and at least one ring-shaped electrode encircling said flexible sleeve;
a plurality of guidelines, each of said guidelines passing through a longitudinal channel of a respective flexible sleeve and having a first end releasably connected to a free end of a respective spline and a second end;
a force application mechanism associated with said support platform, configured to controllably apply and release a tensile force to said second ends of said guidelines such that a spline is flattened when said tensile force is applied to said second end of said respective guideline and reverts to a curved shape when said tensile force is released, so as to enable sliding said flattened spline through said longitudinal channel of said respective flexible sleeve.

2. The apparatus of claim 1, wherein said force application mechanism comprises:
at least one pulley, configured to guide a respective guideline having second end connected to a respective weight; and
a weight control mechanism, configured to control at least downward motion of said weight, such that said guideline applies said tensile force to said free end of said respective spline as said weight descends.

3. The apparatus of claim 1, wherein said support platform is configured to rotate around a central axis.

4. The apparatus of claim 1, wherein said force application mechanism is fixedly coupled to said support platform.

5. The apparatus of claim 1, further comprising a compression mechanism, configured for controllably applying a vertical force to press a top of said expandable basket towards said support platform.

6. The apparatus of claim 1, further comprising a basket support element for supporting a top of said expandable basket while said tensile force is applied.

7. The apparatus of claim 1, wherein said flexible sleeve is configured to contract upon the application of heat, said apparatus further comprising a heat source configured for applying heat to said flexible sleeve so as to secure said flexible sleeve to a spline passing through said flexible sleeve.

8. The apparatus of claim 1, wherein said support platform comprises seats for supporting respective sleeve assemblies.

9. A sub-assembly for manufacture into an end effector of a basket catheter assembly, comprising:
an expandable basket comprising a plurality of curved splines with shape retention;
a plurality of sleeve assemblies, each of said sleeve assemblies respectively comprising a flexible sleeve having a longitudinal channel therethrough and at least one ring-shaped electrode encircling said flexible sleeve; and
a plurality of guidelines threaded through said longitudinal channels of respective sleeve assemblies, each of said guidelines being releasably connected to a free end of a respective spline and configured to apply a tensile force to said free end of said respective spline.

10. The sub-assembly of claim 9, wherein a second end of each of said guidelines is attached to a respective weight.

11. The sub-assembly of claim 9, wherein a second end of each of said guidelines is connected to a force application mechanism configured to apply and release a tensile force on said guidelines.

12. The sub-assembly of claim 9, wherein said splines comprise shape-set nitinol.

13. The sub-assembly of claim 9, wherein said flexible sleeves are configured to contract upon the application of heat.

14. A method of manufacturing an end effector for a basket catheter assembly, comprising:
providing an expandable basket comprising a plurality of curved splines with shape retention;
providing at least one sleeve assembly, each of said sleeve assemblies respectively comprising a flexible sleeve and at least one ring-shaped electrode encircling said flexible sleeve;
releasably connecting a first end of a guideline to a free end of a respective spline through a longitudinal channel of a flexible sleeve, such that pulling on said guideline applies a tensile force to said free end of said respective spline; and
for at least one of said splines:
flattening said spline by applying a tensile force to a second end of a respective guideline;
sliding a longitudinal channel of a respective flexible sleeve onto said flattened spline; and
releasing said tensile force from said second end of said guideline to allow said spline to revert to a curved shape.

15. The method of claim 14, wherein said releasably connecting said guideline to said free end of said at least one spline comprises tying an end of said guideline to a hole in said free end of said spline.

16. The method of claim 14, further comprising:
connecting a second end of said guideline to a weight;
looping said guideline over a pulley while securing said weight against downward motion; and
moving said weight downwards so as to apply said tensile force to said free end of said at least one spline.

17. The method of claim 14, wherein a tensile force is applied simultaneously to at least some of said plurality of splines.

18. The method of claim 14, further comprising positioning said basket on a support platform.

19. The method of claim 18, further comprising controllably applying a downward force on a top of said expandable basket so as to control a position of said top of said expandable basket relative to said support platform.

20. The method of claim 14, further comprising supporting a top of said expandable basket in place while said tensile force is applied.

21. The method of claim 14, wherein a magnitude of said tensile force is sufficient to flatten a spline comprising shape-set nitinol.
